(19) United States
(12) Patent Application Publication
(10) Pub. No.: US 2006/0030525 A1
(43) Pub. Date: Feb. 9, 2006

(54) Apolipoprotein A-I derivatives with altered immunogenicity

(75) Inventors: Shannon Alicia Marshall, San Francisco, CA (US); Gregory Moore, Pasadena, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Xencor, Inc., Monrovia, CA (US)

(21) Appl. No.: 11/189,438

(22) Filed: Jul. 25, 2005

Related U.S. Application Data
(60) Provisional application No. 60/590,689, filed on Jul. 23, 2004.

(51) Int. Cl. A61K 38/17; A61K 31/56; C07K 14/775

(52) U.S. Cl. 514/12; 530/359; 514/171

(57) ABSTRACT

The present invention relates to novel apolipoprotein A-I proteins with altered immunogenicity.

This application claims benefit under 35 USC 119(e) to U.S. Provisional Application No. 60/590,689 filed Jul. 23, 2004, which is incorporated herein by reference in its entirety.

### FIELD OF THE INVENTION

The present invention relates to variant apolipoprotein A-I proteins with reduced immunogenicity. In particular, variants of apolipoprotein A-I with reduced ability to bind one or more human class II MHC molecules are described.

### BACKGROUND OF THE INVENTION

Apolipoprotein A-I (ApoA-I) is a 243-residue lipoprotein consisting of a series of highly homologous 11- and 22-residue repeat units; these repeats have been proposed to form amphipathic α-helices that wrap around and bind lipids creating a belt-like structure that contains aromatic-rich lipid binding domains on its inner surface. ApoA-I is synthesized in the liver and small intestine and is the principle protein component of plasma high-density lipoprotein (HDL); it is also found in chylomicrons. ApoA-I participates with HDL in the reverse transport of cholesterol from tissues to the liver; it promotes cholesterol efflux from peripheral tissues and the arterial wall, activates lecithin cholesterol acyltransferase (LCAT) esterification of HDL-associated cholesterol, and participates in the receptor-mediated delivery of cholesterol ester to the liver. Mechanisms of cholesterol efflux include ABC-1 transport and the scavenger receptor B-1 HDL receptor. In addition to its association with discoidal and spherical HDL, it exists in the plasma in lipid-poor and lipid-free forms. Levels of ApoA-I correlate with HDL's protective effect against atherosclerosis, and overexpression of human ApoA-I inhibits the development of atherosclerosis in transgenic mice and rabbits and in ApoE-deficient mice, suggesting its potential as an anti-atherogenic (see Schultz et al. Nature 365: 762-764 (1993), Warden et al. Science 261: 469-472 (1993), Paszty et al. J Clin Invest 94: 899-903 (1994), both incorporated by reference).

A number of naturally occurring allelic variants of ApoA-I have been characterized. Of special interest are variants that confer enhanced protection from atherosclerosis, including but not limited to the 173C variant of ApoA-I, called ApoA-I Milano, and the 151C variant of ApoA-I, referred to as ApoA-I Paris. The Milano and Paris variants may form disulfide-linked dimers, which may confer improved cholesterol removal, enhanced serum half-life, and reduced heterogeneity of HDL particle size (Chiesa, Ann Med 2003 35: 267-273, incorporated by reference). In early clinical trials, administration of ApoA-I Milano caused regression of vascular plaque in atherosclerotic patients (Nissen et al. Jama 290: 2292-2300 (2003), incorporated by reference).

ApoA-I also lowers LDL cholesterol, and has other activities including acting as an anti-inflammatory, antioxidant, antiviral, antibacterial, and anticoagulant. Potential applications of ApoA-I include use in the prevention or treatment of cardiovascular diseases and diseases associated with lipid or cholesterol homeostasis including atherosclerosis, coronary artery disease, stroke, restenosis, hyperlipidemia, analphalipoproteinemia, hypoalphalipoproteinemia, various forms of amyloidosis, and in viral or bacterial infections. See Brouillette et al. Biochim Biophys Acta 1531: 4-46 (2001), Srinivas et al. Virology 176: 48-57 (1990), Hawkins J Nephrol 16: 443-448 (2003), all incorporated by reference.

Immunogenicity of Apolipoprotein A-I (ApoA-I)

Immunogenicity is a major barrier to the development and utilization of protein therapeutics. Although immune responses are typically most severe for non-human proteins, even therapeutics based on human proteins may be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis.

Several factors can contribute to protein immunogenicity, including but not limited to the protein sequence, the route and frequency of administration, and the patient population.

Immunogenicity may limit the efficacy and safety of a protein therapeutic in multiple ways. Efficacy can be reduced directly by the formation of neutralizing antibodies. Efficacy may also be reduced indirectly, as binding to either neutralizing or non-neutralizing antibodies typically leads to rapid clearance from serum. Severe side effects and even death may occur when an immune reaction is raised. One special class of side effects results when neutralizing antibodies cross-react with an endogenous protein and block its function.

Although in Phase I trials intravenous administration of recombinant ApoA-I Milano has not induced immune responses (Chiesa and Sirtori Curr Opin Lipidol 14: 159-163 (2003), incorporated by reference), ApoA-I, like all proteins, has the potential to induce unwanted immune responses when used as a therapeutic. Accordingly, the development of therapeutics based on ApoA-I may be facilitated by pre-emptively reducing the potential immunogenicity of ApoA-I.

Several methods have been developed to modulate the immunogenicity of proteins. In some cases, PEGylation has been observed to reduce the fraction of patients who raise neutralizing antibodies by sterically blocking access to antibody agretopes (see for example, Hershfield et al. PNAS 1991 88:7185-7189 (1991); Bailon. et al. Bioconjug. Chem. 12: 195-202(2001); He et al. Life Sci. 65: 355-368 (1999), both incorporated by reference). Methods that improve the solution properties of a protein therapeutic may also reduce immunogenicity, as aggregates have been observed to be more immunogenic than soluble proteins.

A more general approach to immunogenicity reduction involves mutagenesis targeted at the agretopes in the protein sequence and structure that are most responsible for stimulating the immune system. Some success has been achieved by randomly replacing solvent-exposed residues to lower binding affinity to panels of known neutralizing antibodies (see for example Laroche et al. Blood 96: 1425-1432 (2000), incorporated by reference). Due to the incredible diversity of the antibody repertoire, mutations that lower affinity to known antibodies will typically lead to production of an another set of antibodies rather than abrogation of immunogenicity. However, in some cases it may be possible to decrease surface antigenicity by replacing hydrophobic and charged residues on the protein surface with polar neutral residues (see Meyer et al. Protein Sci. 10: 491-503 (2001), incorporated by reference).

An alternate approach is to disrupt T-cell activation. Removal of MHC-binding agretopes offers a much more tractable approach to immunogenicity reduction, as the diversity of MHC molecules comprises only ˜10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such agretopes for the purpose of generating less immunogenic proteins has been disclosed previously; see for example WO 98/52976, WO 02/079232, and WO 00/3317, all incorporated by reference.

While mutations in MHC-binding agretopes can be identified that are predicted to confer reduced immunogenicity, most amino acid substitutions are energetically unfavorable. As a result, the vast majority of the reduced immunogenicity sequences identified using the methods described above will be incompatible with the structure and/or function of the protein. In order for MHC agretope removal to be a viable approach for reducing immunogenicity, it is crucial that simultaneous efforts are made to maintain a protein's structure, stability, and biological activity.

There remains a need for novel ApoA-I proteins having reduced immunogenicity. Variants of ApoA-I with reduced immunogenicity could find use in the treatment of a number of ApoA-I responsive conditions.

### SUMMARY OF THE INVENTION

In one aspect, the present invention provides novel apolipoprotein A-I proteins having reduced immunogenicity as compared to naturally occurring ApoA-I proteins. In an additional aspect, the present invention is directed to methods for engineering or designing less immunogenic proteins with ApoA-I activity for therapeutic use.

In one aspect of the present invention, the non-naturally occurring variant ApoA-I protein can include at least two amino acid modifications of a wild-type ApoA-I protein.

In another aspect, the present invention is directed to ApoA-I variants that show decreased binding affinity for one or more class II MHC alleles relative to a parent ApoA-I and which significantly maintain the activity of native naturally occurring ApoA-I.

In a further aspect, the invention provides recombinant nucleic acids encoding the variant ApoA-I proteins, expression vectors, and host cells including the nucleic acids or expression vectors.

In an additional aspect, the invention provides methods of producing a variant ApoA-I protein comprising culturing the host cells of the invention under conditions suitable for expression of the variant ApoA-I protein.

In a further aspect, the invention provides pharmaceutical compositions comprising a variant ApoA-I protein or nucleic acid of the invention and a pharmaceutical carrier.

In a further aspect, the invention provides methods for preventing or treating ApoA-I responsive disorders comprising administering a variant ApoA-I protein or nucleic acid of the invention to a patient.

In an additional aspect, the invention provides methods for screening the class II MHC haplotypes of potential patients in order to identify individuals who are particularly likely to raise an immune response to a wild type or variant ApoA-I therapeutic.

In another aspect, the present invention provides ApoA-I variant proteins comprising amino acid sequences with at least one amino acid insertion, deletion, or substitution compared to the wild type ApoA-I proteins. In one such variation, the present invention is directed to a non-naturally occurring variant ApoA-I protein having reduced immunogenicity as compared with a wild type ApoA-I protein that includes SEQ. ID NO:1. The variant protein includes at least one amino acid modification as compared to the wild type ApoA-I protein. The modification can be of at least one amino acid residue in an agretope selected from among Agretope 1 (residues 17-25), Agretope 2 (residues 44-52), Agretope 3 (residues 47-55), Agretope 4 (residues 82-90), Agretope 5 (residues 148-156), Agretope 6 (residues 159-167), Agretope 7 (residues 170-178), Agretope 8 (residues 214-222), and Agretope 9 (residues 225-233). X₁ and X₂ can each independently be selected from between R and C.

### BRIEF DESCRIPTION OF THE FIGURES

FIG. 1 shows a method for engineering less immunogenic apolipoprotein A-I variants.

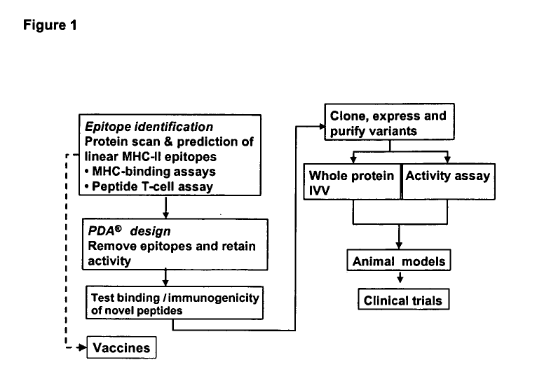

FIG. 2 shows a schematic representation of a method for in vitro testing of the immunogenicity of apolipoprotein A-I peptides or proteins with IVV technology.

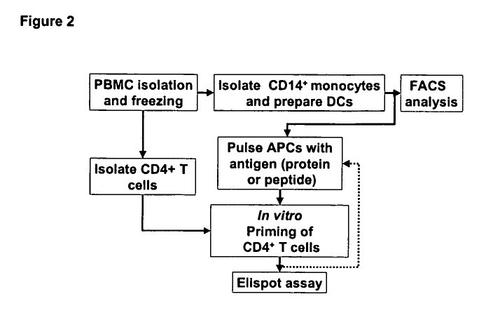

### DETAILED DESCRIPTION OF THE INVENTION

By “nine-mer peptide frame” and grammatical equivalents herein is meant a linear sequence of nine amino acids that is located in a protein of interest. nine-mer frames may be analyzed for their propensity to bind one or more class II MHC alleles. By “allele” and grammatical equivalents herein is meant an alternative form of a gene. Specifically, in the context of class II MHC molecules, alleles comprise all naturally occurring sequence variants of DRA, DRB1, DRB3/4/5, DQA-I, DQB1, DPA-I, and DPB1 molecules. By “ApoA-I-responsive disorders” and grammatical equivalents herein is meant diseases, disorders, and conditions that can benefit from treatment with ApoA-I. Examples of disorders that may benefit from treatment with ApoA-I include, but are not limited to, cardiovascular diseases, particularly atherosclerosis, coronary artery disease, heart attack, stroke, and restenosis, diseases of lipid and cholesterol homeostasis, including conditions associated with ApoA-I or HDL defects or deficiencies, analphalipoproteinemia, hypoalphalipoproteinemia, hyperlipidemia, amyloidosis, and viral and bacterial infections. By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity. By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis. By “reduced immunogenicity” and grammatical equivalents herein is meant a decreased ability to activate the immune system, when compared to the wild type protein. For example, a variant protein can be said to have “reduced immunogenicity” if it elicits neutralizing or non-neutralizing antibodies in lower titer or in fewer patients than the wild type protein. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A variant protein also can be said to have “reduced immunogenicity” if it shows decreased binding to one or more MHC alleles or if it induces T-cell activation in a decreased fraction of patients relative to the parent protein. In a preferred embodiment, the probability of T-cell activation is decreased by at least 5%, with at least 50% or 90% decreases being especially preferred. By “matrix method” and grammatical equivalents thereof herein is meant a method for calculating peptide—MHC affinity in which a matrix is used that contains a score for each possible residue at each position in the peptide, interacting with a given MHC allele. The binding score for a given peptide—MHC interaction is obtained by summing the matrix values for the amino acids observed at each position in the peptide. By “MHC-binding agretopes” and grammatical equivalents herein is meant peptides that are capable of binding to one or more class II MHC alleles with appropriate affinity to enable the formation of MHC—peptide—T-cell receptor complexes and subsequent T-cell activation. MHC-binding agretopes are linear peptide sequences that comprise at least approximately 9 residues. By “parent protein” as used herein is meant a protein that is subsequently modified to generate a variant protein. Said parent protein may be a wild-type or naturally occurring protein, or a variant or engineered version of a naturally occurring protein. “Parent protein” may refer to the protein itself, compositions that comprise the parent protein, or any amino acid sequence that encodes it. Accordingly, by “parent apolipoprotein A-I protein” as used herein is meant an apolipoprotein A-I protein that is modified to generate a variant apolipoprotein A-I protein. By “patient” herein is meant both humans and other animals, particularly mammals, and organisms. Thus the methods are applicable to both human therapy and veterinary applications. In the preferred embodiment the patient is a mammal, and in the most preferred embodiment the patient is human. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992), incorporated by reference], generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized. By “treatment” herein is meant to include therapeutic treatment, as well as prophylactic, or suppressive measures for the disease or disorder. Thus, for example, successful administration of a variant APOA-I protein prior to onset of the disease may result in treatment of the disease. As another example, successful administration of a variant APOA-I protein after clinical manifestation of the disease to combat the symptoms of the disease comprises “treatment” of the disease. “Treatment” also encompasses administration of a variant APOA-I protein after the appearance of the disease in order to eradicate the disease. Successful administration of an agent after onset and after clinical symptoms have developed, with possible abatement of clinical symptoms and perhaps amelioration of the disease, further comprises “treatment” of the disease. By “variant apolipoprotein A-I nucleic acids” and grammatical equivalents is meant nucleic acids that encode variant apolipoprotein A-I proteins. Due to the degeneracy of the genetic code, an extremely large number of nucleic acids may be made, all of which encode the variant apolipoprotein A-I proteins of the present invention, by simply modifying the sequence of one or more codons in a way which does not change the amino acid sequence of the variant apolipoprotein A-I. By “variant apolipoprotein A-I proteins” and grammatical equivalents thereof herein is meant non-naturally occurring apolipoproteinA-I proteins which differ from the wild type, naturally occurring, or parent apolipoprotein A-I protein by at least 1 amino acid insertion, deletion, or substitution. Apolipoprotein A-I variants are characterized by the predetermined nature of the variation, a feature that sets them apart from naturally occurring allelic or interspecies variation of the apolipoprotein A-I protein sequence. The variant apolipoprotein A-I proteins may contain insertions, deletions, and/or substitutions at the N-terminus, C-terminus, or internally. In a preferred embodiment, variant apolipoprotein A-I proteins have at least 1 residue that differs from the naturally occurring apolipoprotein A-I sequence, with at least 2, 3, 4, or 5 different residues being more preferred. Variant apolipoprotein A-I proteins may contain further modifications, for instance mutations that alter stability or solubility or which enable or prevent posttranslational modifications such as PEGylation or glycosylation. Variant apolipoprotein A-I proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels. The apolipoprotein A-I variants typically either exhibit biological activity that is comparable to naturally occurring apolipoprotein A-I or have been specifically engineered to have alternate biological properties. A variant ApoA-I protein has at least one of the following functions: binds lipids via a belt-like structure; participates in reverse transport of cholesterol from tissues to the liver; promotes cholesterol efflux from peripheral tissues and the arterial wall; activates lecithin cholesterol acyltransferase (LCAT) esterification of HDL-associated cholesterol; and participates in the receptor-mediated delivery of cholesterol ester to the liver. Preferably, the variant ApoA-I protein has all the above functions. By “has at least one of the functions” means has at least 50% of the activity of the wild-type protein, or more preferably has 90% of the wild-type activity of the wild-type protein.

By “wild type or wt” and grammatical equivalents thereof herein is meant an amino acid sequence or a nucleotide sequence that is found in nature and includes allelic variations; that is, an amino acid sequence or a nucleotide sequence that has not been intentionally modified. In a preferred embodiment, the wild type sequence includes SEQ_ID NO:1. Alternatively, the wild-type sequence includes a single N-terminal truncation of SEQ ID NO:1, an example of which is disclosed as SEQ ID NO:1 in U.S. Provisional Application No. 60/590,689, incorporated herein by reference.

Identification of MHC-binding Agretopes in Apolipoprotein A-I

MHC-binding peptides are obtained from proteins by a process called antigen processing. First, the protein is transported into an antigen presenting cell (APC) by endocytosis or phagocytosis. A variety of proteolytic enzymes then cleave the protein into a number of peptides. These peptides can then be loaded onto class II MHC molecules, and the resulting peptide-MHC complexes are transported to the cell surface. Relatively stable peptide-MHC complexes can be recognized by T-cell receptors that are present on the surface of naive T cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used to identify agretopes in ApoA-I. Sequence-based information can be used to determine a binding score for a given peptide—MHC interaction (see for example Mallios, Bioinformatics 15: 432-439 (1999); Mallios, Bioinformatics 17: p942-948 (2001); Sturniolo et al. Nature Biotech. 17: 555-561(1999), all incorporated by reference). It is possible to use structure-based methods in which a given peptide is computationally placed in the peptide-binding groove of a given MHC molecule and the interaction energy is determined (for example, see WO 98/59244 and WO 02/069232, both incorporated by reference). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187, incorporated by reference).

In a preferred embodiment, MHC-binding propensity scores are calculated for each 9-residue frame along the apolipoprotein A-I sequence using a matrix method (see Sturniolo et al., supra; Marshall et al., J. Immunol. 154: 5927-5933 (1995), and Hammer et al., J. Exp. Med. 180: 2353-2358 (1994), both incorporated by reference). It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 10 μM or 1 μM affinity.

In a preferred embodiment, the number of hits for each nine-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding agretopes are identified as the nine-mer frames that bind to several class II MHC alleles. In an especially preferred embodiment, MHC-binding agretopes are predicted to bind at least 10 alleles at 5% threshold and/or at least 5 alleles at 1% threshold. Such nine-mer frames may be especially likely to elicit an immune response in many members of the human population.

In a preferred embodiment, MHC-binding agretopes are predicted to bind MHC alleles that are present in at least 0.01-10% of the human population. Alternatively, to treat conditions that are linked to specific class II MHC alleles, MHC-binding agretopes are predicted to bind MHC alleles that are present in at least 0.01-10% of the relevant patient population.

Data about the prevalence of different MHC alleles in different ethnic and racial groups has been acquired by groups such as the National Marrow Donor Program (NMDP); for example see Mignot et al. Am. J. Hum. Genet 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003), all incorporated by reference.

In a preferred embodiment, MHC binding agretopes are predicted for MHC heterodimers comprising highly prevalent MHC alleles. Class II MHC alleles that are present in at least 10% of the US population include but are not limited to: DPA-I*0103, DPA-I*0201, DPB1*0201, DPB1*0401, DPB1*0402, DQA-I*0101, DQA-I*0102, DQA-I*0201, DQA-I*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding agretopes are also predicted for MHC heterodimers comprising moderately prevalent MHC alleles. Class II MHC alleles that are present in 1% to 10% of the US population include but are not limited to: DPA-I*0104, DPA-I*0302, DPA-I*0301, DPB1*0101, DPB1*0202, DPB1*0301, DPB1* 0501, DPB1*0601, DPB1*0901, DPB1*1001, DPB1*1101, DPB1*1301, DPB1*1401, DPB1*1501, DPB1*1701, DPB1*1901, DPB1*2001, DQA-I*0103, DQA-I*0104, DQA-I*0301, DQA-I*0302, DQA-I*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding agretopes may also be predicted for MHC heterodimers comprising less prevalent alleles. Information about MHC alleles in humans and other species can be obtained, for example, from the IMGT/HLA sequence database (ebi.ac.uk/imgt/hla/).

In an especially preferred embodiment, an immunogenicity score is determined for each peptide, wherein said score depends on the fraction of the population with one or more MHC alleles that are hit at multiple thresholds. For example, the equation
Iscore=N(W₁P₁+W₃P₃+W₅P₅)
may be used, where P₁ is the percent of the population hit at 1%, P₃ is the percent of the population hit at 3%, P₅ is the percent of the population hit at 5%, each W is a weighting factor, and N is a normalization factor. In a preferred embodiment, W₁=10, W₃=5, W₅=2, and N is selected so possible scores range from 0 to 100. In this embodiment, agretopes with Iscore greater than or equal to 10 are preferred and agretopes with Iscore greater than or equal to 25 are especially preferred.

In an additional preferred embodiment, MHC-binding agretopes are identified as the nine-mer frames that are located among “nested” agretopes, or overlapping 9-residue frames that are each predicted to bind a significant number of alleles. Such sequences may be especially likely to elicit an immune response.

Preferred MHC-binding agretopes are those agretopes that are predicted to bind, at a 3% threshold, to MHC alleles that are present in at least 5% of the population. Preferred MHC-binding agretopes in apolipoprotein A-I include, but are not limited to, agretope 1: residues 17-25; agretope 2: residues 44-52; agretope 6: residues 159-167; agretope 7: residues 170-178; and agretope 9: residues 225-233.

Especially preferred MHC-binding agretopes are those agretopes that are predicted to bind, at a 1% threshold, to MHC alleles that are present in at least 10% of the population. Especially preferred MHC-binding agretopes in apolipoprotein A-I include, but are not limited to, agretope 6: residues 159-167.

Alternate preferred MHC-binding agretopes are those agretopes that have Iscore greater than or equal to 10. Preferred MHC-binding agretopes in apolipoprotein A-I include, but are not limited to, agretope 1: residues 16-24; agretope 2: residues 43-51; agretope 6: residues 158-166; and agretope 9: residues 224-232.

Alternate especially preferred MHC-binding agretopes are those agretopes that have Iscore greater than or equal to 25. Preferred MHC-binding agretopes in apolipoprotein A-I include, but are not limited to, agretope 6: residues 158-166.

Additional especially preferred MHC-binding agretopes are those agretopes whose sequences partially overlap with additional MHC-binding agretopes. Sets of overlapping MHC-binding agretopes in apolipoprotein A-I include, but are not limited to, residues 43-54.

Confirmation of MHC-Bindinq Agretopes

In a preferred embodiment, the immunogenicity of the above-predicted MHC-binding agretopes is experimentally confirmed by measuring the extent to which peptides comprising each predicted agretope can elicit an immune response. However, it is possible to proceed from agretope prediction to agretope removal without the intermediate step of agretope confirmation.

Several methods, discussed in more detail below, can be used for experimental confirmation of agretopes. For example, sets of naive T cells and antigen presenting cells from matched donors can be stimulated with a peptide containing an agretope of interest, and T-cell activation can be monitored. It is also possible to first stimulate T cells with the whole protein of interest, and then restimulate with peptides derived from the whole protein. If sera are available from patients who have raised an immune response to ApoA-I, it is possible to detect mature T cells that respond to specific epitopes. In a preferred embodiment, interferon gamma or IL-5 production by activated T-cells is monitored using Elispot assays, although it is also possible to use other indicators of T-cell activation or proliferation such as tritiated thymidine incorporation or production of other cytokines.

Patient Genotype Analysis and Screening

HLA genotype is a major determinant of susceptibility to specific autoimmune diseases (see for example Nepom Clin. Immunol. Immunopathol. 67: S50-S55 (1993)) and infections (see for example Singh et al. Emerg. Infect. Dis. 3: 41-49 (1997)), both incorporated by reference. Furthermore, the set of MHC alleles present in an individual can affect the efficacy of some vaccines (see for example Cailat-Zucman et al. Kidney Int. 53: 1626-1630 (1998) and Poland et al. Vaccine 20: 430-438 (2001), both incorporated by reference). HLA genotype may also confer susceptibility for an individual to elicit an unwanted immune response to an apolipoprotein A-I therapeutic.

In a preferred embodiment, class II MHC alleles that are associated with increased or decreased susceptibility to elicit an immune response to ApoA-I proteins are identified. For example, patients treated with ApoA-I therapeutics may be tested for the presence of anti-ApoA-I antibodies and genotyped for class II MHC. Alternatively, T-cell activation assays such as those described above may be conducted using cells derived from a number of genotyped donors. Alleles that confer susceptibility to ApoA-I immunogenicity may be defined as those alleles that are significantly more common in those who elicit an immune response versus those who do not. Similarly, alleles that confer resistance to ApoA-I immunogenicity may be defined as those that are significantly less common in those who do not elicit an immune response versus those that do. It is also possible to use purely computational techniques to identify which alleles are likely to recognize ApoA-I therapeutics.

In one embodiment, the genotype association data is used to identify patients who are especially likely or especially unlikely to raise an immune response to an ApoA-I therapeutic.

Design of Active, Less-Immunogenic Variants

In a preferred embodiment, the above-determined MHC-binding agretopes are replaced with alternate amino acid sequences to generate active variant ApoA-I proteins with reduced or eliminated immunogenicity. Alternatively, the MHC-binding agretopes are modified to introduce one or more sites that are susceptible to cleavage during protein processing. If the agretope is cleaved before it binds to a MHC molecule, it will be unable to promote an immune response. There are several possible strategies for integrating methods for identifying less immunogenic sequences with methods for identifying structured and active sequences, including but not limited to those presented below.

In one embodiment, for one or more nine-mer agretope identified above, one or more possible alternate nine-mer sequences are analyzed for immunogenicity as well as structural and functional compatibility. The preferred alternate nine-mer sequences are then defined as those sequences that have low predicted immunogenicity and a high probability of being structured and active. It is possible to consider only the subset of nine-mer sequences that are most likely to comprise structured, active, less immunogenic variants. For example, it may be unnecessary to consider sequences that comprise highly non-conservative mutations or mutations that increase predicted immunogenicity.

In a preferred embodiment, less immunogenic variants of each agretope are predicted to bind MHC alleles in a smaller fraction of the population than the wild type agretope. In an especially preferred embodiment, the less immunogenic variant of each agretope is predicted to bind to MHC alleles that are present in not more than 5% of the population, with not more than 1% or 0.1% being most preferred.

Substitution Matrices

In another especially preferred embodiment, substitution matrices or other knowledge-based scoring methods are used to identify alternate sequences that are likely to retain the structure and function of the wild type protein. Such scoring methods can be used to quantify how conservative a given substitution or set of substitutions is. In most cases, conservative mutations do not significantly disrupt the structure and function of proteins (see for example, Bowie et al. Science 247: 1306-1310 (1990), Bowie and Sauer Proc. Nat. Acad. Sci. USA 86: 2152-2156 (1989), and Reidhaar-Olson and Sauer Proteins 7: 306-316 (1990), all incorporated by reference). However, non-conservative mutations can destabilize protein structure and reduce activity (see for example, Lim et al. Biochem. 31: 4324-4333 (1992), incorporated by reference). Substitution matrices including but not limited to BLOSUM62 provide a quantitative measure of the compatibility between a sequence and a target structure, which can be used to predict non-disruptive substitution mutations (see Topham et al. Prot. Eng. 10: 7-21 (1997), incorporated by reference). The use of substitution matrices to design peptides with improved properties has been disclosed; see Adenot et al. J. Mol. Graph. Model. 17: 292-309 (1999), incorporated by reference.

Substitution matrices include, but are not limited to, the BLOSUM matrices (Henikoff and Henikoff, Proc. Nat. Acad. Sci. USA 89: 10917 (1992), incorporated by reference, the PAM matrices, the Dayhoff matrix, and the like. For a review of substitution matrices, see for example Henikoff Curr. Opin. Struct. Biol. 6: 353-360 (1996), incorporated by reference. It is also possible to construct a substitution matrix based on an alignment of a given protein of interest and its homologs; see for example Henikoff and Henikoff Comput. Appl. Biosci. 12: 135-143 (1996), incorporated by reference.

In a preferred embodiment, each of the substitution mutations that are considered has a BLOSUM62 score of zero or higher. According to this metric, preferred substitutions include, but are not limited to:

TABLE 1
Conservative mutations

| Wild type | Preferred |
|---|---|
| residue | substitutions |
| A | C S T A G V |
| C | C A |
| D | S N D E Q |
| E | S N D E Q H R K |
| F | M I L F Y W |
| G | S A G N |
| H | N E Q H R Y |
| I | M I L V F |
| K | S N E Q R K |
| L | M I L V F |
| M | Q M I L V F |
| N | S T G N D E Q H R K |
| P | P |
| Q | S N D E Q H R K M |
| R | N E Q H R K |
| S | S T A G N D E Q K |
| T | T A M I L V |
| V | S T A N V |
| W | F Y W |
| Y | H F Y W |

In addition, it is preferred that the total BLOSUM62 score of an alternate sequence for a nine residue MHC-binding agretope is decreased only modestly when compared to the BLOSUM62 score of the wild type nine residue agretope. In a preferred embodiment, the score of the variant nine-mer is at least 50% of the wild type score, with at least 67%, 75%, 80%, or 90% being especially preferred.

Alternatively, alternate sequences can be selected that minimize the absolute reduction in BLOSUM score; for example it is preferred that the score decrease for each nine-mer is less than 20, with score decreases of less than about 10 or about 5 being especially preferred. The exact value may be chosen to produce a library of alternate sequences that is experimentally tractable and also sufficiently diverse to encompass a number of active, stable, less immunogenic variants.

In a preferred embodiment, substitution mutations are preferentially introduced at positions that are substantially solvent exposed. As is known in the art, solvent exposed positions are typically more tolerant of mutation than positions that are located in the core of the protein.

In a preferred embodiment, substitution mutations are preferentially introduced at positions that are not highly conserved. As is known in the art, positions that are highly conserved among members of a protein family are often important for protein function, stability, or structure, while positions that are not highly conserved often may be modified without significantly impacting the structural or functional properties of the protein.

Alanine Substitutions

In an alternate embodiment, one or more alanine substitutions may be made, regardless of whether an alanine substitution is conservative or non-conservative. As is known in the art, incorporation of sufficient alanine substitutions may be used to disrupt intermolecular interactions.

In a preferred embodiment, variant nine-mers are selected such that residues that have been or can be identified as especially critical for maintaining the structure or function of apolipoprotein A-I retain their wild type identity. In alternate embodiments, it may be desirable to produce variant ApoA-I proteins that do not retain wild type activity. In such cases, residues that have been identified as critical for function may be specifically targeted for modification.

Most data indicate that a variety of apolipoprotein sequences are capable of promoting cholesterol efflux from the plasma membrane, though ApoA-I is more effective than ApoA-I I, Apo C or Apo E (Brouillette et al. Biochim Biophys Acta 1531: 4-46 (2001). In-vivo expression of ApoA-I mutant proteins has shown that both the N- and C-terminal domains are important for lipid association as well as for the esterification reaction, particularly binding of cholesteryl esters and formation of mature α-migrating lipoproteins. Activation of LCAT requires interaction with the central helix 6 and interaction with the ATP binding cassette transporter A-I requires the C-terminal domain, which is proposed to mediate the opening of the helix bundle formed by lipid-free or lipid-poor ApoA-I and allow its association with hydrophobic binding sites. For details on specific deletions and point mutations and their impact on structure and function see Brouillette et al. Biochim Biophys Acta 1531: 446 (2001), and Marcel and Kiss Curr Opin Lipidol 14: 151-157 (2003), both incorporated by reference.

Several variants of ApoA-I have been shown to result in various clinical forms of amyloidosis, characterized by deposits of amyloid fibrils composed predominantly of β-sheet structure. Eleven amyloidogenic variants are known, eight of which are single amino acid substitutions, two are deletions and one a deletion/insertion (Hawkins J Nephrol 16: 443-448 (2003), incorporated by reference). As amyloid formation is associated with a number of pathologies, in a preferred embodiment such variants are not selected for therapeutic use.

Two human alleles of ApoA-I, Milano (173C) and Paris (151C) result in a deficiency of HDL but also decrease coronary artery disease. These variants are found in vivo in a disulfide cross-linked form, either as homodimers or as heterodimer with ApoA-II; they provide longer serum half-life and better cholesterol removal, and appear to reduce the heterogeneity of HDL particle size (Chiesa and Sirtori Ann Med 35: 267-273 (2003), incorporated by reference). In a preferred embodiment, ApoA-I Milano, ApoA-I Paris, or other variant with enhanced protection from atherosclerosis is selected for further optimization and therapeutic use.

Protein Design Methods

Protein design methods and MHC agretope identification methods may be used together to identify stable, active, and minimally immunogenic protein sequences (see WO03/006154, incorporated by reference). The combination of approaches provides significant advantages over the prior art for immunogenicity reduction, as most of the reduced immunogenicity sequences identified using other techniques fail to retain sufficient activity and stability to serve as therapeutics.

Protein design methods may identify non-conservative or unexpected mutations that nonetheless confer desired functional properties and reduced immunogenicity, as well as identifying conservative mutations. Nonconservative mutations are defined herein to be all substitutions not included in Table 1 above; nonconservative mutations also include mutations that are unexpected in a given structural context, such as mutations to hydrophobic residues at the protein surface and mutations to polar residues in the protein core.

Furthermore, protein design methods may identify compensatory mutations. For example, if a given first mutation that is introduced to reduce immunogenicity also decreases stability or activity, protein design methods may be used to find one or more additional mutations that serve to recover stability and activity while retaining reduced immunogenicity. Similarly, protein design methods may identify sets of two or more mutations that together confer reduced immunogenicity and retained activity and stability, even in cases where one or more of the mutations, in isolation, fails to confer desired properties.

A wide variety of methods are known for generating and evaluating sequences. These include, but are not limited to, sequence profiling (Bowie and Eisenberg, Science 253(5016): 164-70, (1991)), residue pair potentials (Jones, Protein Science 3: 567-574, (1994)), and rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)), all incorporated by reference.

Protein Design Automation (PDA®) Technology

In an especially preferred embodiment, rational design of improved apolipoprotein A-I variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all incorporated by reference.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

PDA® utilizes three-dimensional structural information. In a most preferred embodiment, the structure of a type I interferon is determined using X-ray crystallography or NMR methods, which are well known in the art. The crystal structure of the N-terminal truncated human ApoA-I (residues 43-243) and the NMR structure of intact human ApoA-I have been solved; see Borhani et al. Proc Natl Acad Sci U S A 94: 12291-12296 (1997), Borhani et al. Acta Crystallogr D Biol Crystallogr 55 (Pt 9): 1578-1583 (1999), Okon et al. FEBS Lett 517: 139-143 (2002), all incorporated by reference. In an alternate embodiment, a homology model is built, using methods known to those in the art.

In a preferred embodiment, the results of matrix method calculations are used to identify which of the 9 amino acid positions within the agretope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each agretope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an agretope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDA® technology calculations. In this way, it is possible to select sequences that are active and less immunogenic in a single computational step.

Combining Immunogenicity Reduction Strategies

In a preferred embodiment, more than one method is used to generate variant proteins with desired functional and immunological properties. For example, substitution matrices may be used in combination with PDA® technology calculations. Strategies for immunogenicity reduction include, but are not limited to, those described in U.S. Ser. No. 09/903,378; WO 01/21823; U.S. Ser. No. 10/039,170; WO 02/00165; U.S. Ser. No. 10/339,788; 10/754,296; and U.S. Ser. No. 10/638,995, all incorporated by reference.

In a preferred embodiment, a variant protein with reduced binding affinity for one or more class II MHC alleles is further engineered to confer improved solubility. As protein aggregation may contribute to unwanted immune responses, increasing protein solubility may reduce immunogenicity. See U.S. Ser. No. 10/820,467, filed Mar. 30, 2004, entitled, “Interferon Variants With Improved Properties”, incorporated by reference.

In an additional preferred embodiment, a variant protein with reduced binding affinity for one or more class II MHC alleles is further modified by derivitization with PEG or another molecule. As is known in the art, PEG may sterically interfere with antibody binding or improve protein solubility, thereby reducing immunogenicity. In an especially preferred embodiment, rational PEGylation methods are used. See, PCT/US2004/008425; and U.S. Ser. No. 10/10/956,352, filed Sep. 30, 2004, entitled, “Rational Chemical Modification,” both incorporated by reference.

In a preferred embodiment, PDA® technology and matrix method calculations are used to remove more than one MHC-binding agretope from a protein of interest.

Generating the Variants

Variant interferon nucleic acids and proteins of the invention may be produced using a number of methods known in the art.

Preparing Nucleic Acids Encoding the ApoA-I Variants

In a preferred embodiment, nucleic acids encoding ApoA-I variants are prepared by total gene synthesis, or by site-directed mutagenesis of a nucleic acid encoding wild type or variant ApoA-I protein. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et at. Biotechniques 30: 249-252 (2001), all incorporated by reference).

Expression Vectors

In a preferred embodiment, an expression vector that comprises the components described below and a gene encoding a variant ApoA-I protein is prepared. Numerous types of appropriate expression vectors and suitable regulatory sequences for a variety of host cells are known in the art. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

The expression vector may include a secretory leader sequence or signal peptide sequence that provides for secretion of the variant ApoA-I protein from the host cell. Suitable secretory leader sequences that lead to the secretion of a protein are known in the art. The signal sequence typically encodes a signal peptide comprised of hydrophobic amino acids, which direct the secretion of the protein from the cell. The protein is either secreted into the growth media or, for prokaryotes, into the periplasmic space, located between the inner and outer membrane of the cell. For expression in bacteria, bacterial secretory leader sequences, operably linked to a variant APOA-I encoding nucleic acid, are usually preferred.

Transfection/Transformation

The variant ApoA-I nucleic acids are introduced into the cells either alone or in combination with an expression vector in a manner suitable for subsequent expression of the nucleic acid. The method of introduction is largely dictated by the targeted cell type. Exemplary methods include CaPO₄ precipitation, liposome fusion, Lipofectin®, electroporation, viral infection, dextran-mediated transfection, polybrene mediated transfection, protoplast fusion, direct microinjection, etc. The variant APOA-I nucleic acids may stably integrate into the genome of the host cell or may exist either transiently or stably in the cytoplasm. As outlined herein, a particularly preferred method utilizes retroviral infection, as outlined in PCT/US97/01019, incorporated by reference.

Hosts for the Expression of ApoA-I Variants

Appropriate host cells for the expression of ApoA-I variants include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc (see the ATCC cell line catalog, hereby expressly incorporated by reference), as well as primary cell lines.

ApoA-I variants can also be produced in more complex organisms, including but not limited to plants (such as corn, tobacco, and algae) and animals (such as chickens, goats, cows); see for example Dove, Nature Biotechnol. 20: 777-779 (2002), incorporated by reference.

In one embodiment, the cells may be additionally genetically engineered, that is, contain exogenous nucleic acid other than the expression vector comprising the variant ApoA-I nucleic acid.

Expression and Purification Methods

The variant ApoA-I proteins of the present invention are produced by culturing a host cell transformed with an expression vector containing nucleic acid encoding a variant ApoA-I protein, under the appropriate conditions to induce or cause expression of the variant ApoA-I protein. The conditions appropriate for variant ApoA-I protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

In a preferred embodiment, the ApoA-I variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, an ApoA-I variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, N.Y., 3d ed. (1994), incorporated by reference. The degree of purification necessary will vary depending on the desired use, and in some instances no purification will be necessary. For further references on purification of type I interferons, see for example Moschera et al. Meth. Enzym. 119: 177-183 (1986); Tarnowski et al. Meth. Enzym. 119:153-165(1986); Thatcher et al. Meth. Enzym. 119:166-177 (1986); Lin et al. Meth. Enzym. 119:183-192 (1986), all incorporated by reference. Methods for purification of apolipoprotein A-I are disclosed in U.S. Pat. No. 4,462,940 and U.S. Pat. No. 4,894, 330, both incorporated by reference.

Protocols for the expression and purification of ApoA-I have been disclosed for bacteria such as E. coli (prepro, pro, and mature forms), baculovirus such as Sf9, and mammalian cells. See for example Pyle et al. Biochemistry 35: 12046-12052 (1996), Sorci-Thomas et al. J Lipid Res 37: 673-683 (1996), Pyle et al. Anal Biochem 253: 253-258 (1997), Schmidt et al. Protein Expr Purif 10: 226-236 (1997), Panagotopulos et al. Protein Expr Purif 25: 353-361 (2002), Ryan et al. Protein Expr Purif 27: 98-103 (2003), all incorporated by reference.

Assaying the Activity of the Variants

In a preferred embodiment, the wild-type and variant proteins are analyzed for biological activities by suitable methods known in the art.

Lipid binding assays include analysis of the ability to form HDL-like bilayer disk complexes with dimyristoyl phosphatidylcholine (DMPC), and studies on DMPC binding kinetics and size of HDL particles formed (Gorshkova et al. Biochemistry 41: 10529-10539 (2002), Fang et al. Biochemistry 42: 13260-13268 (2003), both incorporated by reference. Other in vitro assays include activation of lethicin cholesterol acyltransferase (LCAT) (Vanloo et al. Biochim Biophys Acta 1128: 258-266 (1992), Datta et al. J Lipid Res 42: 1096-1104 (2001), Martin-Campos et al. J Lipid Res 43: 115-123 (2002), all incorporated by reference, measurement of ³H-cholesterol efflux from macrophages (Lin et al. J Lipid Res 40: 1618-1627 (1999), Major et al. Arterioscler Thromb Vasc Biol 21: 1790-1795 (2001), both incorporated by reference), LDL-uptake in cultured human liver cells, and oxidized LDL-mediated cholesterol and cholesterol ester accumulation in cultured human vascular smooth muscle cells.

In vivo assays include inhibition of foam cell formation, cholesterol accumulation in atherosclerotic lesions in ApoE-deficient mice, (Boisvert et al. Arterioscler Thromb Vasc Biol 19: 525-530 (1999), incorporated by reference), effects on plasma cholesterol, HDL, and atherosclerotic plaques in ApoE-knockout mice expressing the ApoA-I transgene (Paszty et al. J Clin Invest 94: 899-903 (1994), incorporated by reference), determination of atheromas in a rabbit model of carotid focal lesion (Chiesa et al. Circ Res 90: 974-980 (2002), Chiesa et al. Curr Opin Lipidol 14: 159-163 (2003), both incorporated by reference, and prevention of lipoxygenase-mediated oxidation of phospholipids (Bielicki and Oda Biochemistry 41: 2089-2096 (2002), incorporated by reference).

Determining the Immunogenicity of the Variants

In a preferred embodiment, the immunogenicity of the apolipoprotein A-I variants is determined experimentally to confirm that the variants do have reduced or eliminated immunogenicity relative to the parent protein.

In a preferred embodiment, ex vivo T-cell activation assays are used to experimentally quantitate immunogenicity. In this method, antigen presenting cells and naive T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et al. J. Immunol. Meth., 24: 17-24 (2000), incorporated by reference).

Other suitable T-cell assays include those disclosed in Meidenbauer, et al. Prostate 43, 88-100 (2000); Schultes, B. C and Whiteside, T. L., J. Immunol. Methods 279, 1-15 (2003); and Stickler, et al., J. Immunotherapy, 23, 654-660 (2000), all incorporated by reference.

In a preferred embodiment, the PBMC donors used for the above-described T-cell activation assays will comprise class II MHC alleles that are common in patients requiring treatment for apolipoprotein A-I responsive disorders. For example, for most diseases and disorders, it is desirable to test donors comprising all of the alleles that are prevalent in the population. However, for diseases or disorders that are linked with specific MHC alleles, it may be more appropriate to focus screening on alleles that confer susceptibility to apolipoprotein A-I responsive disorders.

In a preferred embodiment, the MHC haplotype of PBMC donors or patients that raise an immune response to the wild type or variant apolipoprotein A-I are compared with the MHC haplotype of patients who do not raise a response. This data may be used to guide preclinical and clinical studies as well as aiding in identification of patients who will be especially likely to respond favorably or unfavorably to the apolipoprotein A-I therapeutic.

In an alternate preferred embodiment, immunogenicity is measured in transgenic mouse systems. For example, mice expressing fully or partially human class II MHC molecules may be used.

In an alternate embodiment, immunogenicity is tested by administering the apolipoprotein A-I variants to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans. Similarly, genetically engineered mouse models expressing human MHC peptide-binding domains may be used (see for example Sonderstrup et al. Immunol. Rev. 172: 335-343 (1999) and Forsthuber et al. J. Immunol. 167: 119-125 (2001), both incorporated by reference).

Formulation and Administration to Patients

Once made, the variant ApoA-I proteins and nucleic acids of the invention find use in a number of applications. In a preferred embodiment, a variant ApoA-I protein or nucleic acid is administered to a patient to treat an ApoA-I related disorder.

The administration of the variant ApoA-I proteins of the present invention, preferably in the form of a sterile aqueous solution, may be done in a variety of ways, including, but not limited to, orally, parenterally, subcutaneously, intravenously, intranasally, transdermally, intraperitoneally, intramuscularly, intrapulmonary, vaginally, rectally, intranasally or intraocularly. In some instances, the variant ApoA-I protein may be directly applied as a solution or spray. Depending upon the manner of introduction, the pharmaceutical composition may be formulated in a variety of ways.

The pharmaceutical compositions of the present invention comprise a variant ApoA-I protein in a form suitable for administration to a patient. In the preferred embodiment, the pharmaceutical compositions are in a water-soluble form, such as being present as pharmaceutically acceptable salts, which is meant to include both acid and base addition salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant ApoA-I proteins are added in a micellular formulation; see U.S. Pat. No. 5,833,948.

Combinations of pharmaceutical compositions may be administered. Moreover, the compositions may be administered in combination with other therapeutics.

ApoA-I Milano/phospholipid complex administered intravenously for 5 doses at weekly intervals produced significant regression of coronary atherosclerosis in patients as measured by intravascular ultrasound (Nissen et al. Jama 290: 2292-2300 (2003), incorporated by reference).

In a preferred embodiment, the nucleic acid encoding the variant APOA-I proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. The oligonucleotides may be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993), incorporated by reference). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), both incorporated by reference. For review of gene marking and gene therapy protocols see Anderson et al., Science 256:808-813 (1992), incorporated by reference.

While the foregoing invention has been described above, it will be clear to one skilled in the art that various changes and additional embodiments made be made without departing from the scope of the invention. All publications, patents, patent applications (provisional, utility and PCT) or other documents cited herein are incorporated by references in their entirety.

### EXAMPLES

Example 1

### Identification of MHC-Binding Agretopes in Apolipoprotein A-I

Matrix method calculations (Sturniolo, supra) were conducted using the parent apolipoprotein A-I sequence shown in SEQ_ID:1, X139C, X174C. Calculations were also repeated on the ApoA-I Milano (SEQ_ID:1,) and ApoA-I Paris (SEQ_ID:3) variants.

Agretopes were predicted for the following alleles, each of which is present in at least 1% of the US population: DRB1*0101, DRB1*0102, DRB1*0301, DRB1*0401, DRB1*0402, DRB1*0404, DRB1*0405, DRB1*0408, DRB1*0701, DRB1*0801, DRB1*1101, DRB1*1102, DRB1*1104, DRB1*1301, DRB1*1302, DRB1*1501, and DRB1*1502.

For each nine-mer that is predicted to bind to at least one allele at a 5% threshold, the number of alleles that are hit at 1%, 3%, and 5% thresholds were given, as well as the percent of the US population that are predicted to react to the nine-mer. The worst nine-mers are shown in bold. They are predicted to be immunogenic in at least 10% of the US population, using a 1% threshold.

TABLE 2

Predicted MHC-binding agretopes in apolipoprotein A-I.
The immunogenicity score, number of alleles, and percent
of population hit at 1%, 3%, and 5% thresholds are shown.
Especially preferred agretopes are predicted to affect at
least 10% of the population, using a 1% threshold.

| | | | | | | | | | |
|---|---|---|---|---|---|---|---|---|---|
| Agretope | | | | 1% | 3% | 5% | 1% | 3% | 5% |
| number | Residues | Sequence | IScore | hits | hits | hits | pop | pop | pop |

| | | | | | | | | | |
|---|---|---|---|---|---|---|---|---|---|
| Agretope 1 | 17-25 | VYVDVLKDS | 13.08 | 0 | 1 | 1 | 0% | 21% | 21% |
| Agretope 2 | 44-52 | LKLLDNWDS | 19.06 | 3 | 5 | 9 | 9% | 12% | 38% |
| Agretope 3 | 47-55 | LDNWDSVTS | 0.42 | 0 | 0 | 1 | 0% | 0% | 2% |
| Agretope 4 | 82-90 | LRQEMSKDL | 4.50 | 0 | 0 | 2 | 0% | 0% | 18% |
| Agretope 5 | 148-156 | MRDRARAHV | 1.25 | 0 | 0 | 1 | 0% | 0% | 5% |
| Agretope 6 | 159-167 | LRTHLAPYS | 32.86 | 2 | 5 | 6 | 12% | 38% | 47% |
| Agretope 7 | 170-178 | LRQRLAARL | 3.13 | 0 | 1 | 1 | 0% | 5% | 5% |
| Agretope 8 | 214-222 | LRQGLLPVL | 5.23 | 0 | 0 | 1 | 0% | 0% | 21% |
| Agretope 9 | 225-233 | FKVSFLSAL | 15.17 | 0 | 1 | 1 | 0% | 25% | 25% |

ApoA-I Milano has an increased Iscore (13.32 vs. 3.13) for Agretope 7 and is otherwise predicted to have the same MHC agretopes as ApoA-I. ApoA-I Paris has a decreased Iscore (0.00 vs. 1.25) for Agretope 5 and is otherwise predicted to have the same MHC agretopes as ApoA-I.

Alleles that are predicted as “hits” for each of the agretopes above are shown in the table below. “1” indicates a hit using a 1% threshold, “3” indicates a hit using a 3% threshold, and “5” indicates a hit using a 5% threshold.

TABLE 3
Predicted MHC-binding agretopes in apolipoprotein A-I. DRB1 alleles
that are predicted to bind to each allele at 1%, 3%, and 5% cutoffs are marked
with “1”, “3”, or “5”, respectively.

| Agretope | | | | | | | | | | | | | | | | | |
|---|---|---|---|---|---|---|---|---|---|---|---|---|---|---|---|---|---|
| number | 101 | 102 | 301 | 401 | 402 | 404 | 405 | 408 | 701 | 801 | 1101 | 1102 | 1104 | 1301 | 1302 | 1501 | 1502 |
| Agretope 1 | — | — | 3 | — | — | — | — | — | — | — | — | — | — | — | — | — | — |
| Agretope 2 | 10 | 5 | — | 5 | 1 | 1 | 3 | 1 | — | — | 10 | 3 | 5 | 5 | 10 | 10 | 10 |
| Agretope 3 | — | — | — | — | 5 | — | — | — | — | — | — | — | — | — | — | — | — |
| Agretope 4 | 10 | 5 | — | 5 | — | — | — | — | 10 | — | — | — | — | — | — | — | — |
| Agretope 5 | — | — | — | — | — | — | — | — | — | 5 | — | — | — | 10 | — | — | — |
| Agretope 6 | 10 | 10 | 3 | 5 | 3 | — | — | — | — | — | 10 | 1 | 10 | 1 | 3 | 10 | — |
| Agretope 7 | — | 10 | — | — | 10 | — | — | — | — | 3 | — | — | — | — | — | 10 | — |
| Agretope 8 | — | — | 5 | — | — | — | — | — | — | — | — | — | — | — | — | 10 | — |
| Agretope 9 | — | — | — | — | — | — | — | — | 3 | — | — | — | — | — | — | — | — |

### Example 2

### Identification of Suitable Less Immunogenic Sequences for MHC-Bindinq Agretopes in Apolipoprotein A-I as Determined by BLOSUM Method

MHC-binding agretopes that were predicted to bind alleles present in at least 10% of the US population, using a 1% threshold, were analyzed to identify suitable less immunogenic variants.

At each agretope, all possible combinations of amino acid substitutions were considered, with the following requirements: (1) each substitution has a score of 0 or greater in the BLOSUM62 substitution matrix, (2) each substitution is capable of conferring reduced binding to at least one of the MHC alleles considered, and (3) once sufficient substitutions are incorporated to prevent any allele hits at a 1% threshold, no additional substitutions are added to that sequence.

Alternate sequences were scored for immunogenicity and structural compatibility. Preferred alternate sequences were defined to be those sequences that are not predicted to bind to any of the 17 MHC alleles tested above using a 1% threshold, and that have a total BLOSUM62 score that is at least 85% of the wild type score.

TABLE 4

Suitable less immunogenic variants of agretope 6
(residues 159-167). B(wt) is the BLOSUM62 score
of the wild type nine-mer, I(alt) is the percent
of the US population containing one or more MHC
alleles that are predicted to bind the alternate
nine-mer at a 1% threshold, and B(alt) is the
BLOSUM62 score of the alternate nine-mer.

| | | | | | |
|---|---|---|---|---|---|
| Sequence ID | Variant sequence | I(alt) | B(alt) | WT sequence | B(wt) |

| | | | | | |
|---|---|---|---|---|---|
| SEQ_ID:4 | LNTHLAPYS | 0 | 43 | LRTHLAPYS | 48 |
| SEQ_ID:5 | LETHLAPYS | 0 | 43 | LRTHLAPYS | 48 |
| SEQ_ID:6 | LQTHLAPYS | 0 | 44 | LRTHLAPYS | 48 |
| SEQ_ID:7 | LHTHLAPYS | 0 | 43 | LRTHLAPYS | 48 |
| SEQ_ID:8 | LKTHLAPYS | 0 | 45 | LRTHLAPYS | 48 |
| SEQ_ID:9 | LRTNLAPYS | 0 | 41 | LRTHLAPYS | 48 |
| SEQ_ID:10 | LRTELAPYS | 0 | 40 | LRTHLAPYS | 48 |
| SEQ_ID:11 | LRTQLAPYS | 0 | 40 | LRTHLAPYS | 48 |
| SEQ_ID:12 | LRTRLAPYS | 0 | 40 | LRTHLAPYS | 48 |
| SEQ_ID:13 | LRTYLAPYS | 0 | 42 | LRTHLAPYS | 48 |
| SEQ_ID:14 | LRTHLGPYS | 0 | 44 | LRTHLAPYS | 48 |
| SEQ_ID:15 | LRTHLAPYT | 0 | 45 | LRTHLAPYS | 48 |
| SEQ_ID:16 | LRTHLAPYA | 0 | 45 | LRTHLAPYS | 48 |
| SEQ_ID:17 | LRTHLAPYG | 0 | 44 | LRTHLAPYS | 48 |
| SEQ_ID:18 | LRTHLAPYN | 0 | 45 | LRTHLAPYS | 48 |
| SEQ_ID:19 | LRTHLAPYD | 0 | 44 | LRTHLAPYS | 48 |
| SEQ_ID:20 | LRTHLAPYE | 0 | 44 | LRTHLAPYS | 48 |
| SEQ_ID:21 | LRTHLAPYK | 0 | 44 | LRTHLAPYS | 48 |
| SEQ_ID:22 | FRTHLAPYQ | 0 | 40 | LRTHLAPYS | 48 |

### Example 3

### Identification of Suitable Less Immunogenic Sequences for MHC-Binding Agretopes in Apolipoprotein A-I as Determined by PDA® Technology

Each position in the agretopes of interest was analyzed to identify a subset of amino acid substitutions that are potentially compatible with maintaining the structure and function of the protein. PDA® technology calculations were run for each position of each nine-mer agretope and compatible amino acids for each position were saved. In these calculations, side-chains within 5 Angstroms of the position of interest were permitted to change conformation but not amino acid identity. The variant agretopes were then analyzed for immunogenicity. The PDA® energies and Iscore values for the wild-type nine-mer agretope were compared to the variants and the subset of variant sequences with lower predicted immunogenicity and PDA® energies within 5.0 kcal/mol of the wild-type (wt) were noted.

TABLE 5
Suitabie less immunogenic
variants of agretope 6 (residues 159-167).
E(PDA) is the energy determined using
PDA ® technology calculations compared
against the wild-type, Iscore: Anchor is
the Iscore for the agretope, and Iscore:
Overlap is the sum of the Iscores for all of
the overlapping agretopes.

| | | Iscore | Iscore |
|---|---|---|---|
| Var. | E(PDA) | Anchor | Overlap |
| wt | 0.00 | 32.85 | 0.00 |
| L159Y | −1.10 | 29.12 | 0.00 |
| L159F | 0.32 | 29.12 | 0.00 |
| L159K | 2.95 | 0.00 | 0.00 |
| L159R | 3.97 | 0.00 | 0.00 |
| L159T | 4.09 | 0.00 | 0.00 |
| L159Q | 4.42 | 0.00 | 0.00 |
| L159N | 4.90 | 0.00 | 0.00 |
| R160V | 0.21 | 26.52 | 0.00 |
| R160K | 0.51 | 3.57 | 0.00 |
| R160Q | 0.92 | 3.57 | 0.00 |
| R160I | 1.19 | 3.57 | 0.00 |
| R160A | 1.26 | 0.00 | 0.00 |
| R160M | 1.33 | 3.57 | 0.00 |
| R160L | 1.37 | 2.95 | 0.00 |
| R160N | 1.44 | 0.42 | 0.00 |
| R160G | 1.47 | 0.00 | 0.00 |
| R160T | 1.62 | 0.00 | 0.00 |
| R160E | 1.75 | 0.00 | 0.00 |
| R160H | 1.78 | 0.42 | 0.00 |
| R160D | 2.07 | 0.00 | 0.00 |
| R160S | 2.12 | 0.00 | 0.00 |
| R160Y | 2.22 | 0.42 | 0.00 |
| R160W | 2.35 | 0.00 | 0.00 |
| R160F | 2.52 | 0.42 | 0.00 |
| T161E | 0.01 | 2.95 | 0.00 |
| T161D | 0.35 | 0.42 | 0.00 |
| H162W | −0.75 | 9.25 | 0.00 |
| H162R | −0.52 | 2.04 | 0.00 |
| H162V | −0.45 | 22.28 | 0.00 |
| H162K | −0.33 | 10.29 | 0.00 |
| H162Y | −0.20 | 18.38 | 0.00 |
| H162F | −0.06 | 30.23 | 0.00 |
| H162Q | 0.85 | 20.16 | 0.00 |
| H162E | 1.38 | 9.66 | 0.00 |
| H162I | 1.77 | 29.51 | 0.00 |
| H162D | 1.95 | 28.87 | 0.00 |
| H162A | 1.96 | 18.72 | 0.00 |
| H162S | 2.04 | 26.17 | 0.00 |
| H162T | 3.77 | 0.00 | 0.00 |
| H162G | 4.14 | 13.08 | 0.00 |
| A164Y | 0.12 | 5.87 | 0.00 |
| A164E | 0.24 | 0.42 | 0.00 |
| A164D | 0.34 | 0.00 | 0.00 |
| A164L | 0.52 | 30.04 | 0.00 |
| A164Q | 0.56 | 17.44 | 0.00 |
| A164H | 0.79 | 23.71 | 0.00 |
| A164G | 0.99 | 13.76 | 0.00 |
| A164F | 1.21 | 0.42 | 0.00 |
| A164M | 2.07 | 3.57 | 0.00 |
| A164W | 2.34 | 0.42 | 0.00 |
| P165R | 0.39 | 26.04 | 0.00 |
| P165A | 0.46 | 32.80 | 0.00 |
| P165T | 0.58 | 19.37 | 0.00 |
| P165Y | 0.89 | 27.92 | 0.00 |
| P165G | 1.22 | 8.19 | 0.00 |
| P165K | 1.47 | 32.80 | 0.00 |
| P165Q | 1.47 | 28.91 | 0.00 |
| P165S | 1.71 | 18.33 | 0.00 |
| P16SD | 1.91 | 11.68 | 0.00 |
| P165E | 1.99 | 16.87 | 0.00 |
| P165W | 2.60 | 26.04 | 0.00 |
| S167I | −4.83 | 30.72 | 0.00 |
| S167V | −3.42 | 16.17 | 0.00 |
| S167H | −2.97 | 1.46 | 0.00 |
| S167M | −2.57 | 29.90 | 0.00 |
| S167F | −2.44 | 15.61 | 0.00 |
| S167L | −2.44 | 12.24 | 0.00 |
| S167T | −2.03 | 1.23 | 0.00 |
| S167Q | −1.94 | 2.51 | 0.00 |
| S167A | −1.92 | 5.00 | 0.00 |
| S167G | −1.03 | 8.42 | 0.00 |
| S167R | −0.47 | 12.67 | 0.00 |
| S167K | −0.12 | 1.04 | 0.00 |
| S167E | −0.07 | 0.00 | 0.00 |
| S167N | 0.11 | 0.00 | 0.00 |
| S167D | 1.30 | 0.00 | 0.00 |
| S167Y | 2.38 | 6.45 | 0.00 |
| S167P | 2.38 | 0.00 | 0.00 |
| S167W | 2.87 | 0.00 | 0.00 |

The amino acid sequence of a wild type human apolipoprotein A-I protein (SEQ_ID:1), GenBank gi|4557321|ref|NP_{—}000030.1|[4557321], is shown below. The residues corresponding to the mature peptide are included. In the first polymorphism, X₁ and X₂ are R. In the Paris polymorphism, X₁ (position 151) is C. In the Milano polymorphism, X₂ (position 173) is C.

ApoA-I

(SEQ_ID:1)

DEPPQSPWDRVKDLATVYVDVLKDSGRDYVSQFEGSALGKQLNLKLLDNW
DSVTSTFSKLREQLGPVTQEFWDNLEKETEGLRQEMSKDLEEVKAKVQPY
LDDFQKKWQEEMELYRQKVEPLRAELQEGARQKLHELQEKLSPLGEEMRD
X¹ARAHVDALRTHLAPYSDELRQX₂LAARLEALKENGGARLAEYHAKATE
HLSTLSEKAKPALEDLRQGLLPVLESFKVSFLSALEEYTKKLNTQ

We claim:

1. A non-naturally occurring variant ApoA-I protein having reduced immunogenicity as compared to a wild type ApoA-I protein comprising SEQ. ID NO:1, wherein said variant protein comprises at least two amino acid modifications.

2. A non-naturally occurring variant ApoA-I protein having reduced immunogenicity as compared to a wild type ApoA-I protein comprising SEQ. ID NO:1, said variant protein comprising at least one amino acid modification as compared to said wild type ApoA-I protein, wherein said modification is of at least one amino acid residue in an agretope selected from the group consisting of: Agretope 1 (residues 17-25), Agretope 2 (residues 44-52), Agretope 3 (residues 47-55), Agretope 4 (residues 82-90), Agretope 5 (residues 148-156), Agretope 6 (residues 159-167), Agretope 7 (residues 170-178), Agretope 8 (residues 214-222), and Agretope 9 (residues 225-233), and wherein X₁ and X₂ are each independently selected from the group consisting of R or C.

3. A non-naturally occurring variant ApoA-I protein of claim 2, wherein the modification is of at least one residue in Agretope 1 (residues 17-25).

4. A non-naturally occurring variant ApoA-I protein of claim 2, wherein the modification is of at least one residue in Agretope 2 (residues 44-52).

5. A non-naturally occurring variant ApoA-I protein of claim 2, wherein the modification is of at least one residue in Agretope 3 (residues 47-55).

6. A non-naturally occurring variant ApoA-I protein of claim 2, wherein the modification is of at least one residue in Agretope 4 (residues 82-90).

7. A non-naturally occurring variant ApoA-I protein of claim 2, wherein the modification is of at least one residue in Agretope 5 (residues 148-156).

8. A non-naturally occurring variant ApoA-I protein of claim 2, wherein the modification is of at least one residue in Agretope 6 (residues 159-167).

9. A non-naturally occurring variant ApoA-I protein of claim 8, wherein at least one modification is selected from the group consisting of residues 159, 160, 161, 162, 164, 165, and 167; the residue at position 159 is selected from the group consisting of L, Y, F, K, R, T, Q, and N; the residue at position 160 is selected from the group consisting of R, V, K, Q, I, A, M, L, N, G, T, E, H, D, S, Y, W, and F; the residue at position 161 is selected from the group consisting of T, E and D; the possible modifications at position 162 are selected from the group consisting of H, Y, F, K, R, T, Q, N; the residue at position 164 is selected from the group consisting of A, Y, E, D, L, Q, H, G, F, M, and W; the residue at position 165 is selected from the group consisting of P, R, A, T, Y, G, K, Q, S, D, E, and W; and the residue at position 167 is selected from the group consisting of S, I, V, H, M, F, L, T, Q, A, G, R, K, E, N, D, Y, P, and W.

10. A non-naturally occurring variant ApoA-I protein of claim 9, wherein at least one modification is selected from the group consisting of residues 159, 160, 162, 164, and 167; wherein the residue at position 159 is selected from the group consisting of L, K, R, T, Q, and N; the residue at position 160 is selected from the group consisting of A, G, T, E, D, S, and W; the residue at position 162 is H or T; the residue at position 164 is A or D; and the residue at position 167 is selected from the group consisting of S, E, N, D, P, and W.

11. A non-naturally occurring variant ApoA-I protein of one of claims 1 or 10, wherein X₂ is C.

12. A non-naturally occurring variant ApoA-I protein of one of claims 1 or 10, wherein X₁ is C.

13. A non-naturally occurring variant ApoA-I protein of claim 2, wherein the modification is of at least one residue in Agretope 7 (residues 170-178).

14. A non-naturally occurring variant ApoA-I protein of claim 2, wherein the modification is of at least one residue in Agretope 8 (residues 214-222).

15. A non-naturally occurring variant ApoA-I protein of claim 2, wherein the modification is of at least one residue in Agretope 9 (residues 225-233).


SEQUENCE LISTING (23 sequences)

SEQ ID NO: 1 - protein, 243 aa, Artificial
  Synthetic
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDXARAHVDALRTHLAPYSDELRQXLAARLEALKENGGARLAEYHAKATEHLSTLSEKAKPALEDLRQGLLPVLESFKVSFLSALEEYTKKLNTQ

SEQ ID NO: 2 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 5 - protein, 9 aa, Artificial
  Synthetic
LNTHLAPYS

SEQ ID NO: 6 - protein, 9 aa, Artificial
  Synthetic
LETHLAPYS

SEQ ID NO: 7 - protein, 9 aa, Artificial
  Synthetic
LQTHLAPYS

SEQ ID NO: 8 - protein, 9 aa, Artificial
  Synthetic
LHTHLAPYS

SEQ ID NO: 9 - protein, 9 aa, Artificial
  Synthetic
LKTHLAPYS

SEQ ID NO: 10 - protein, 9 aa, Artificial
  Synthetic
LRTNLAPYS

SEQ ID NO: 11 - protein, 9 aa, Artificial
  Synthetic
LRTELAPYS

SEQ ID NO: 12 - protein, 9 aa, Artificial
  Synthetic
LRTQLAPYS

SEQ ID NO: 13 - protein, 9 aa, Artificial
  Synthetic
LRTRLAPYS

SEQ ID NO: 14 - protein, 9 aa, Artificial
  Synthetic
LRTYLAPYS

SEQ ID NO: 15 - protein, 9 aa, Artificial
  Synthetic
LRTHLGPYS

SEQ ID NO: 16 - protein, 9 aa, Artificial
  Synthetic
LRTHLAPYT

SEQ ID NO: 17 - protein, 9 aa, Artificial
  Synthetic
LRTHLAPYA

SEQ ID NO: 18 - protein, 9 aa, Artificial
  Synthetic
LRTHLAPYG

SEQ ID NO: 19 - protein, 9 aa, Artificial
  Synthetic
LRTHLAPYN

SEQ ID NO: 20 - protein, 9 aa, Artificial
  Synthetic
LRTHLAPYD

SEQ ID NO: 21 - protein, 9 aa, Artificial
  Synthetic
LRTHLAPYE

SEQ ID NO: 22 - protein, 9 aa, Artificial
  Synthetic
LRTHLAPYK

SEQ ID NO: 23 - protein, 9 aa, Artificial
  Synthetic
FRTHLAPYQ